(19) United States
(12) Patent Application Publication
(10) Pub. No.: US 2003/0228587 A1
(43) Pub. Date: Dec. 11, 2003

(54) Nucleic acid molecule encoding IWU-1

(76) Inventors: J.H. David Wu, Pittsford, NY (US); Takeshi Omasa, Nishinomiya (JP); Yi-Guang Chen, Bar Harbor, ME (US); Athanassios Mantalaris, Harrow (GB)

Correspondence Address: Michael L. Goldman, Esq., NIXON PEABODY LLP, Clinton Square, P.O. Box 31051, Rochester, NY, 14603-1051, US

(21) Appl. No.: 10/352,272

(22) Filed: Jan. 27, 2003

Related U.S. Application Data
(60) Provisional application No. 60/351,933, filed on Jan. 25, 2002.

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.2

(57) ABSTRACT

The present invention relates to an isolated nucleic acid molecule encoding an IWU-1 protein or polypeptide; an isolated IWU-1 protein or polypeptide encoded by the nucleic acid molecule of the present invention; an isolated antibody against the protein or polypeptide encoded by the nucleic acid molecule; and uses for the nucleic acid molecule, the protein, and its antibody, including in the making of a vaccine, and in various in methods, including for detection of IWU-1 in a biological sample; treating a disease condition in a subject; regulating IWU-1 expression in a subject, and in methods of screening drugs that regulate IWU-1 expression. Also disclosed are vectors, host cells, and transgenic animals having the nucleic acid molecule encoding an IWU-1 protein or polypeptide of the present invention.

This application claims the benefit of U.S. Provisional Patent Application Serial No. 60/351,933, filed Jan. 25, 2002.

The subject matter of this application was made with support from the United States Government under The National Science Foundation, Grant No. BES-9631670 and NASA, Grant No. NAG 8-1382. The U.S. Government may have certain rights.

### FIELD OF THE INVENTION

The present invention relates to a nucleic acid molecule encoding an IWU-1 gene, an IWU-1 protein or polypeptide, and uses thereof.

### BACKGROUND OF THE INVENTION

Cancer is a complex and devastating group of diseases that kills one in five adults in developing countries. Although cancers arise from a wide variety of cells and tissues in the body, there are unifying features of this group of diseases. Cancer is predominantly a genetic disease, resulting from the accumulation of mutations that promote clonal selection of cells that exhibit uncontrolled growth and division. For example, by the time a tumor reaches a palpable size of about one centimeter, it has already undergone about thirty cell doublings, has a mass of approximately one gram, and contains about one billion malignant cells. The result of such uncontrolled growth of tumor cells is the formation of disorganized tissue that compromises the function of normal organs, ultimately threatening the life of the patient. Obviously, methods for prevention, early detection, and effective treatment of cancer are of paramount importance.

The disruption of external or internal regulation of cellular growth leading to uncontrolled cell proliferation can occur at many levels and, indeed, does occur at multiple levels in most tumors. Further, although tumor cells can no longer control their own proliferation, they still must use the same basic cellular machinery employed by normal cells to drive their growth and replication.

Research on the mechanistic basis of carcinogenesis has resulted in a refined understanding of the molecular nature of genetic changes that initiate tumor formation. Specific genes have been identified that are frequently mutated in tumor cells. A few key genes have been identified that are very commonly mutated in a large number of different tumors, such as the oncogene ras and the tumor suppressor genes p53 and Rb. Furthermore, genes that are mutated in tumor cells tend to have functions that cluster in one of the following categories: DNA repair, chromosomal integrity, cell cycle control, growth factor signaling, apoptosis, differentiation, angiogenesis, immune response, and cell migration.

It is becoming widely recognized that small (30-130 amino acid) single-span membrane proteins have important regulatory roles in cells. Among these are Isk, which modulates the activity of KVLQT1 and HERG cardiac K⁺ channels. (Sweadner et al., “The FXYD Gene Family of Small Ion Transport Regulators or Channels: cDNA Sequence, Protein Signature Sequence, and Expression, Genomic 68:41-56 (2000)). The ATP1γ1/PLMNMAT8 family proteins (Chen et al., “Characterization of the Human and Rat Phospholemman (PLM) cDNA and Localization of the Human PLM Gene to Chromosome 19q13.1,” Genomics 41:435-443 (1997); PRO-SITE accession No. PS01310), including ATP1γ1 (Na⁺+K⁺-ATPase γ-subunit), chloride conductance inducer protein MAT8 (Mammary Tumor, 8 kDa), CHIF (channel inducer factor), PLM (phospholemman), and mouse protein RIC (related to ion channels), are a group of proteins which seem to serve as chloride or potassium channels or channel regulators. These proteins, except RIC which consists of 178 amino acids, are very small, their sizes ranging from 58 amino acids for ATP1γ1 to 92 amino acids for PLM. These proteins are structurally characterized by the presence of a single putative transmembrane domain, but little is known of their biological roles. However, because ion channels are involved in many critical cellular processes, and have been implicated by researchers in the origin or treatment of many diseases (Aidley et al., Ion Channels—Molecules in Action Cambridge Univ Press (1996); Cell Physiology Source Book Second Edition, N. Sperelakis, ed., Academic Press (1997)), what is needed now is the identification and characterization of an exemplary member of this protein family at the genetic

Research on the mechanistic basis of carcinogenesis has resulted in a refined understanding of the molecular nature of genetic changes that initiate tumor formation. Specific genes have been identified that are frequently mutated in tumor cells. A few key genes have been identified that are very commonly mutated in a large number of different tumors, such as the oncogene ras and the tumor suppressor genes p53 and Rb. Furthermore, genes that are mutated in tumor cells tend to have functions that cluster in one of the following categories: DNA repair, chromosomal integrity, cell cycle control, growth factor signaling, apoptosis, differentiation, angiogenesis, immune response, and cell migration.

It is becoming widely recognized that small (30-130 amino acid) single-span membrane proteins have important regulatory roles in cells. Among these are Isk, which modulates the activity of KVLQT1 and HERG cardiac K⁺ channels. (Sweadner et al., “The FXYD Gene Family of Small Ion Transport Regulators or Channels: cDNA Sequence, Protein Signature Sequence, and Expression, Genomic 68:41-56 (2000)). The ATP1γ1/PLM/MAT8 family proteins (Chen et al., “Characterization of the Human and Rat Phospholemman (PLM) cDNA and Localization of the Human PLM Gene to Chromosome 19q13.1,” Genomics 41:435-443 (1997); PRO-SITE accession No. PS01310), including ATP1γ1 (Na⁺+K⁺-ATPase γ-subunit), chloride conductance inducer protein MAT8 (Mammary Tumor, 8 kDa), CHIF (channel inducer factor), PLM (phospholemman), and mouse protein RIC (related to ion channels), are a group of proteins which seem to serve as chloride or potassium channels or channel regulators. These proteins, except RIC which consists of 178 amino acids, are very small, their sizes ranging from 58 amino acids for ATP1γ1 to 92 amino acids for PLM. These proteins are structurally characterized by the presence of a single putative transmembrane domain, but little is known of their biological roles. However, because ion channels are involved in many critical cellular processes, and have been implicated by researchers in the origin or treatment of many diseases (Aidley et al., Ion Channels—Molecules in Action Cambridge Univ Press (1996); Cell Physiology Source Book Second Edition, N. Sperelakis, ed., Academic Press (1997)), what is needed now is the identification and characterization of an exemplary member of this protein family at the genetic level for use in the detection and prevention of, and as treatment for, disease conditions that arise when these important regulatory molecules become dysfunctional.

The present invention is directed to overcoming these and other deficiencies in the art.

### SUMMARY OF THE INVENTION

The present invention relates to an isolated nucleic acid molecule encoding an IWU-1 protein or polypeptide, wherein the nucleic acid molecule either: 1) has a nucleotide sequence of SEQ ID NO: 1; 2) encodes a protein or polypeptide having an amino acid sequence of SEQ ID NO: 2; 3) has a nucleotide sequence that is at least 55% similar to the nucleotide sequence of SEQ ID NO: 1 by basic BLAST using default parameters analysis, or 4) hybridizes to the nucleotide sequence of SEQ ID NO: 1 under stringent conditions characterized by a hybridization buffer comprising 5×SSC at a temperature of 55° C.

The present invention also relates to an isolated IWU-1 protein or polypeptide, wherein the protein or polypeptide has an amino acid sequence of SEQ ID NO: 2 or includes a motif corresponding to DXFXYDXXSLRXXG.

Another aspect of the present invention is a double-stranded RNA molecule is transcribed from the nucleic acid molecule of the present invention.

Another aspect of the present invention is a method of detecting the expression of IWU-1 in a biological sample. This method involves providing an antibody or binding portion thereof which recognizes the IWU-1 polypeptide or protein of the present invention; contacting the antibody or binding portion thereof with the biological sample under conditions effective to bind an antibody or binding portion thereof with any IWU-1 protein or polypeptide present in the sample; and detecting any binding that occurs between the antibody or binding portion thereof and the biological sample, thereby indicating the expression of IWU-1 in the sample.

The present invention also relates to a second method of detecting IWU-1 expression in a biological sample. This method involves providing the nucleic acid molecule of the present invention as a probe or primer in a nucleic acid hybridization assay; contacting the sample with the probe or primer under conditions effective to permit formation of a complex of the probe or primer and any nucleic acid molecule which hybridizes to the probe or primer; and detecting formation of complex in the sample, thereby indicating a presence of IWU-1 expression in the sample.

The present invention also relates to a third method of detecting IWU-1 expression in a biological sample. This method involves providing a nucleic acid molecule of the present invention encoding an IWU-1 protein or polypeptide as a probe or primer in a gene amplification detection procedure; contacting the sample with the probe or primer under conditions effective to amplify probe or primer-specific nucleic acid molecules; and detecting any amplified probe or primer-specific molecules, thereby indicating a presence of IWU-1 in the sample.

Another aspect of the present invention is a method of treating a disease condition in a subject. This method involves providing a nucleic acid molecule according to the present invention or a probe thereto; contacting the nucleic acid molecule or probe thereto with a cell or tissue sample of a subject under conditions effective to bind to cells overexpressing IWU-1 from the cell or tissue sample; and removing cells or tissues which are selected by the nucleic acid molecule or probe thereto, thereby treating the disease condition.

The present invention also relates to another method of treating a disease condition in a subject. This method involves providing an antibody or binding portion thereof that recognizes the IWU-1 polypeptide or protein of the present invention; contacting the antibody or binding portion thereof with a cell or tissue sample of the subject under conditions effective to bind the antibody or binding portion thereof to cells overexpressing IWU-1 from the cell or tissue sample; and removing cells or tissues which bind to the antibody or binding portion thereof, thereby treating the disease condition.

Another aspect of the present invention is a third method of treating a disease condition in a subject. This method involves providing a therapeutic amount of a pharmaceutical conjugate having an antibody or binding portion thereof against the protein or polypeptide of the present invention and a cytotoxic component; and administering the conjugate to a subject under conditions effective to form an immune complex with any IWU-1 protein or polypeptide, thereby treating the disease condition.

Another aspect of the present invention is a vaccine having a carrier, and an IWU-1 polypeptide or protein or antigenic fragment thereof.

Another aspect of the present invention is a fourth method of treating a disease condition in a subject. This method involves providing a composition having an IWU-1 polypeptide or protein or antigenic fragment thereof; and administering a therapeutically effective amount of the composition to a subject, thereby treating the disease condition.

Another aspect of the present invention is a method of regulating IWU-1 expression in a subject. This method involves administering to the subject the antisense nucleic acid molecule which is complementary to the nucleic acid molecule of the present invention.

Another aspect of the present invention is a second method of regulating IWU-1 expression in a subject. This method involves administering to the subject a double stranded RNA molecule transcribed from the nucleic acid molecule of the present invention.

Another aspect of the present invention is a method of gene therapy for treating a disease condition. This method involves administering to a subject the nucleic acid molecule of the present invention, thereby treating the disease condition of the subject.

The present invention also relates to a transgenic animal having an altered expression of IWU-1, where the altered expression results from the introduction of a nucleic acid molecule of the present invention into the animal.

Another aspect of the present invention is a method of screening drugs that regulate IWU-1 activity. This method involves providing the IWU-1 protein or polypeptide of the present invention; providing a reagent upon which IWU-1 exerts activity; providing a test compound; combining the IWU-1 protein or polypeptide, the reagent, and the test compound in a mixture; determining the activity of IWU-1 upon the reagent in the mixture; and measuring any difference between the activity of IWU-1 upon the reagent with and without the test compound.

Another aspect of the present invention is a method of screening drugs that regulate IWU-1 expression. This method involves transforming a host cell with a nucleic acid molecule encoding an IWU-1 protein or polypeptide; culturing the transformed cells; adding a test compound to the isolated cells; and determining whether the test compound regulates expression of IWU-1 in the transformed cells.

Another aspect of the present invention is another method of screening drugs that regulate IWU-1 expression. This method involves isolating cells from a transgenic animal having an altered expression of IWU-1, wherein the altered expression results from the introduction of a nucleic acid molecule of the present invention into the animal; adding a test compound to the culture containing the transformed cells; and determining whether the test compound regulates expression of IWU-1 in the transformed cells.

### BRIEF DESCRIPTION OF THE DRAWINGS

FIG. 1 shows the nucleotide sequence encoding IWU-1 and its deduced amino acid sequence (GenBank accession No. AF177940), and their location in the EST clone A1358167. The IWU-1 cDNA consists of the open reading frame at nucleotide positions 19-365 (SEQ ID NO: 1) within the full-length 647 bp sequence of EST A1358167 (SEQ ID NO: 15). The 115 amino acid sequence of the IWU-1 protein (SEQ ID NO: 2) encoded by the IWU-1 cDNA is also shown in FIG. 1. The stop codons in frame with the putative open reading frame are marked by asterisks. The putative transmembrane domain is underlined. The sequence derived from the cDNA fragment, band 6-6, is between positions 122 and 392. The remaining sequence was derived from the EST clone AI358167.

FIG. 2 shows the protein sequence homology among IWU-1 and the ATP1γ1/PLM/MAT-8 family proteins within the region containing the transmembrane domain. The ATP1γ1PLM/MAT-8 family consensus sequence DXFXYDXXSLRXXG (SEQ ID NO: 16) (PROSITE Accession No. PS01310) is shown on the top. The amino acids in the IWU-1 protein matching the consensus sequence are bolded. Dashed lines indicate amino acids identical to those of IWU-1. The GenBank accession numbers are: AF177940 (IWU-1) (SEQ ID NO: 3), P97808 (RIC) (SEQ ID NO: 4); AAB09425 (ATP1γ1) (SEQ ID NO: 5); CAA63604 (MAT8) (SEQ ID NO: 6); AAC51286 (PLM) (SEQ ID NO: 7); and Q63113 (CHIF) (SEQ ID NO: 8).

FIG. 3 shows the protein sequence homology among IWU-1 and the cytoplasmic regions of the human type 1A (AT_{1A}R) and type 1B (AT_{2B}R) angiotensin II receptors. The bold residues in the AT_{1A}R and AT_{1B}R sequences represent the conserved amino acids among different species (i.e., human, bovine, rat, mouse, turkey, and Xenopus) (Takayanagi et al., “Molecular Cloning, Sequence Analysis and Expression of a cDNA Encoding Human Type-1 Angiotensin II Receptor,” Biochem. Biophys. Res. Commun. 183:910-916 (1992)). The space represents a gap. The identical residues and favored substitutions (+) are shown at the bottom. The GenBank accession numbers are: AF177940 (IWU-1) (SEQ ID NO: 9); P30556 (AT_{1A}R) (SEQ ID NO: 10); and Q13725 (AT_{1B}R) (SEQ ID NO: 11).

FIG. 4 is a schematic diagram depicting IWU-1 and the related protein sequences. The hatched box represents the region containing the putative transmembrane domain of the ATP1γ1/PLM/MAT-8 family proteins shown in FIG. 2. The box with vertical lines indicates the additional homologous region between IWU-1 and mouse RIC. The checkered box represents the homologous region among IWU-1 and the AT_{1A}R/AT_{1B}R cytoplasmic region shown in FIG. 3.

FIG. 5 shows the expression of IWU-1 in human bone marrow. A band of the expected size (342 bp; arrow) was obtained as the RT-PCR product using a human bone marrow (BM) cDNA library as the template and primers specific to the IWU-1 gene. M=DNA molecular weight markers.

### DETAILED DESCRIPTION OF THE INVENTION

The present invention relates to an isolated nucleic acid molecule encoding an IWU-1 protein or polypeptide. One form of the nucleic acid molecule of the present invention is identified herein as IVU-1, and has a nucleotide sequence corresponding to SEQ ID NO: 1, as follows:

| | |
|---|---|
| atgtgggaga ggggtttctt ccagatgcag actctcagta atatcccttg tttctgcctc | 60 |
| catggctcac tcctgccctc cactgatctg gccactctct cagctcatcc cactgatgac | 120 |
| accacgacgc tctctgagag accatcccca agcacagacg tccagacaga cccccagacc | 180 |
| ctcaagccat ctggttttca tgaggatgac cccttcttct atgatgaaca caccctccgg | 240 |
| aaacgggggc tgttggtcgc agctgtgctg ttcatcacag gcatcatcat cctcaccagt | 300 |
| ggcaagtgca ggcagctgtc ccggttatgc cggaatcatt gcaggtg 347 | |

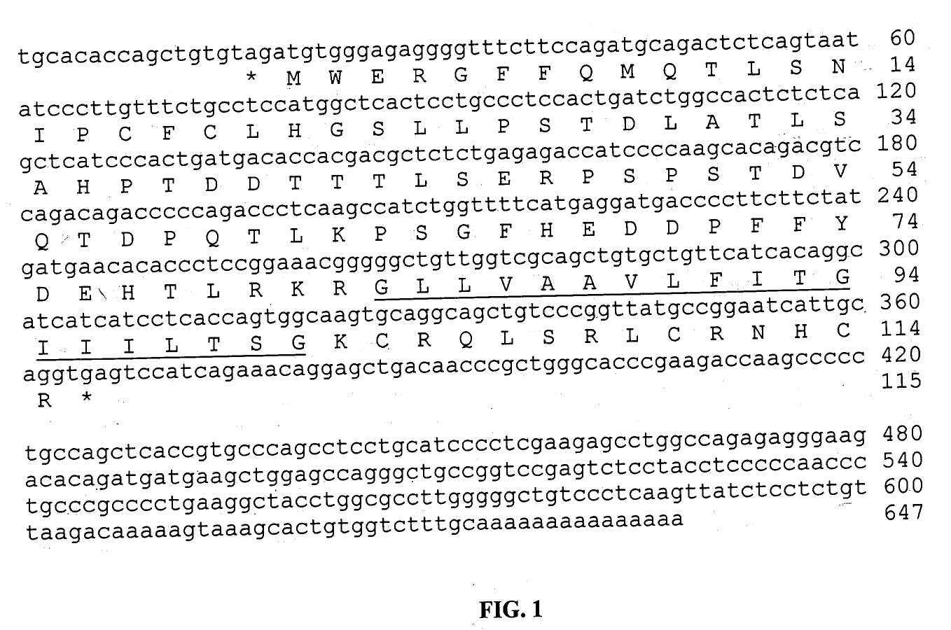

IWU-1, cloned from human bone marrow, is a cDNA molecule having 347 nucleotides, including the open reading frame for the IWU-1 protein and a stop codon. IWU-1 is shown in FIG. 1 in relation to its location within the full-length 647 bp sequence of EST A1358167 (SEQ ID NO: 15), from which it was isolated. The nucleic acid sequence corresponding to SEQ ID NO: 1 encodes a protein or polypeptide identified herein as IWU-1, which has a deduced amino acid sequence corresponding to SEQ ID NO: 2, as follows:

```
Met Trp Gln Arg Gly Phe Phe Gln Met Gln Thr Leu
    1 5 10
Ser Asn Ile Pro Cys Phe Cys Leu His Gly Ser Leu
                  15 20
Leu Pro Ser Thr Asp Leu Ala Thr Leu Ser Ala His
  25 30 35
Pro Thr Asp Asp Thr Thr Thr Leu Ser Glu Arg Pro
                          40 45
Ser Pro Ser Thr Asp Val Gln Thr Asp Pro Gln Thr
          50 55 60
Len Lys Pro Ser Gly Phe His Glu Asp Asp Pro Phe
65 70
Phe Tyr Asp Glu His Thr Leu Arg Lys Arg Gly Leu
                  75 80
Leu Val Ala Ala Val Leu Phe Ile Thr Gly Ile Ile
  85 90 95
Ile Leu Thr Ser Gly Lys Cys Arg Gln Leu Ser Arg
                          100 105
Leu Cys Arg Asn His Cys Arg
        110 115
```

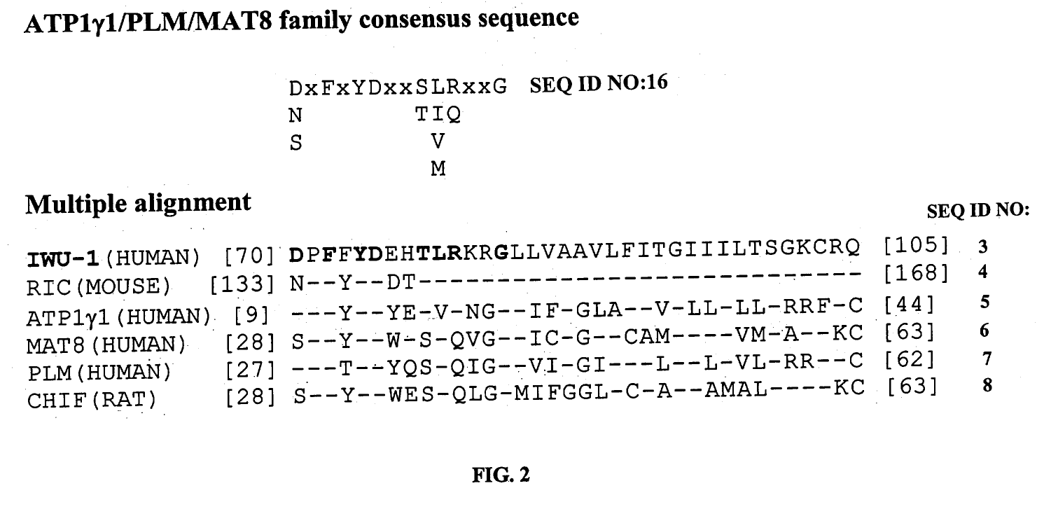

IWU-1 is a protein of 115 amino acids with a calculated molecular mass of 12.9 kDa. The deduced amino acid sequence exhibits high homology (>68%) to members of the ATP1γ1/PLM/MAT8 family of single transmembrane proteins, primarily in the region containing the putative transmembrane domain. The sequence at the amino-terminal side exhibits high homology (>61%) to the cytoplasmic region of the angiotensin II type 1 receptors. Thus, the isolated nucleic acid molecule of the present invention encodes an ion channel protein or ion regulator having a transmembrane domain and an angiotensin-homologous domain. Also included in the protein or polypeptide of the present invention is the ATP1γ1/PLM/MAT-8 family consensus sequence DXFXYDXXSLRXXG (SEQ ID NO: 16), as shown in FIG. 2. Members of the ATP1γ1/PLM/MAT-8 family are known to be expressed in tumor cells, for example, in human breast tumors and human breast tumor cell lines (Morrison et al., “MAT-8, A Novel Phospholemman-like Protein Expressed in Human Breast Tumors, Induces a Chloride Conductance in Xenopus Oocytes,” J. Biol Chem 270(5): 2176-2182 (1995), which is hereby incorporated by reference in its entirety); are up-regulated in epithelial cells by oncogenic Ras and Neu (Fu et al., “E2a-Pbx1 Induces Aberrant Expression of Tissue-Specific and Developmentally Regulated Genes When Expressed in NIH 3T3 Fibroblasts,” Mol Cell Biol 17: 1503-1512 (1997); Morrison et al., “Neu and Ras Initiate Murine Mammary Tumors That Share Genetic Markers Generally Absent in C-myc and Int-2-Initiated Tumors,” Oncogene 9:3417-3426 (1994), which are hereby incorporated by reference in their entirety).

Also suitable as an isolated nucleic acid molecule according to the present invention is a nucleic acid molecule having a nucleotide sequence that is at least 55% similar to the nucleotide sequence of SEQ ID NO: 1 by basic BLAST using default parameters analysis. Also suitable as an isolated nucleic acid molecule according to the present invention is an isolated nucleic acid molecule encoding an IWU-1 protein, wherein the nucleic acid hybridizes to the nucleotide sequence of SEQ ID NO: 1 under stringent conditions characterized by a hybridization buffer comprising 5×SSC at a temperature of 45° C. More stringent conditions, such as hybridization at 52° C. or 55° C. are also suitable. Other examples of high stringency conditions include: 4-5×SSC/0.1% w/v SDS at 54° C. for 1-3 hours; hybridization at 4×SSC at 65° C., followed by a washing in 0.1×SSC at 65° C. for about one hour. Alternatively, an exemplary stringent hybridization condition is in 50% formamide, 4×SSC, at 42° C. Still another example of stringent conditions include hybridization at 62° C. in 6×SSC, 0.05×BLOTTO, and washing at 2×SSC, 0.1% SDS at 62° C. The skilled artisan is aware of various parameters which may be altered during hybridization and washing and which will either maintain or change the stringency conditions. For the purposes of defining the level of stringency, reference can conveniently be made to Sambrook et al., Molecular Cloning: A Laboratory Manual, Third Edition, Cold Spring Harbor: Cold Spring Harbor Laboratory Press, New York (2001)(which is hereby incorporated by reference in its entirety).

The IWU-1 protein or polypeptide of the present invention is preferably produced in purified form by conventional techniques. Typically, the protein or polypeptide of the present invention is secreted into the growth medium of recombinant E. coli. To isolate the protein, the E. coli host cell carrying a recombinant plasmid is propagated, homogenized, and the homogenate is centrifuged to remove bacterial debris. The supernatant is then subjected to sequential ammonium sulfate precipitation. The fraction containing the protein of the present invention is subjected to gel filtration in an appropriately sized dextran or polyacrylamide column to separate the proteins. If necessary, the protein fraction may be further purified by HPLC. Alternative methods may be used as suitable.

Mutations or variants of the above polypeptide or protein are encompassed by the present invention. Variants may be modified by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of the above protein are also encompassed by the present invention. Suitable fragments can be produced by several means. In the first, subclones of the gene encoding the protein of the present invention are produced by conventional molecular genetic manipulation by subcloning gene fragments. The subclones then are expressed in vitro or in vivo in bacterial cells to yield a smaller protein or peptide.

In another approach, based on knowledge of the primary structure of the protein of the present invention, fragments of the gene of the present invention may be synthesized by using the PCR technique together with specific sets of primers chosen to represent particular portions of the protein. These then are cloned into an appropriate vector for increased expression of an accessory peptide or protein.

Chemical synthesis can also be used to make suitable protein fragments. Such a synthesis is carried out using known amino acid sequence for the protein of the present invention. These fragments can then be separated by conventional procedures (e.g., chromatography, SDS-PAGE) and used in the methods of the present invention.

The present invention also relates to the expression of the IWU-1 protein or polypeptide in cells. Generally, this involves inserting the nucleic acid molecule into an expression system to which the nucleic acid molecule is heterologous (i.e., not normally present). The vector contains the necessary elements for the transcription and translation of the inserted protein-coding sequences. In preparing the nucleic acid constructs of the present invention, the various nucleic acid molecules of the present invention may be inserted or substituted into a bacterial plasmid-vector. Any convenient plasmid may be employed, which will be characterized by having a bacterial replication system, a marker which allows for selection in a bacterium and generally one or more unique, conveniently located restriction sites. Numerous plasmids, referred to as transformation vectors, are available for transformation. Suitable vectors include, but are not limited to, the following: viral vectors, such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK ± or KS ± (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference in its entirety), and any derivatives thereof. The selection of a vector will depend on the preferred transformation technique and target cells for transfection.

Certain “control elements” or “regulatory sequences” are also incorporated into the plasmid-vector constructs of the present invention. These include non-transcribed regions of the vector and 5′ and 3′ untranslated regions, which interact with host cellular proteins to carry out transcription and translation. Such elements may vary in their strength and specificity. Depending on the vector system and host utilized, any number of suitable transcription and/or translation elements, including constitutive, inducible, and repressible promoters, as well as minimal 5′ promoter elements may be used.

A constitutive promoter is a promoter that directs constant expression of a gene in a cell. Examples of some constitutive promoters that are widely used for inducing expression of transgenes include the nopoline synthase (“NOS”) gene promoter, from Agrobacterium tumefaciens (U.S. Pat. No. 5,034,322 issued to Rogers et al., which is hereby incorporated by reference in its entirety), the cauliflower mosaic virus (“CaMV”) 35S and 19S promoters (U.S. Pat. No. 5,352,605 issued to Fraley et al., which is hereby incorporated by reference in its entirety), those derived from any of the several actin genes, which are known to be expressed in most cells types (U.S. Pat. No. 6,002,068 issued to Privalle et al., which is hereby incorporated by reference in its entirety), and the ubiquitin promoter (“ubi”), which is the promoter of a gene product known to accumulate in many cell types. Examples of constitutive promoters for use in mammalian cells include the RSV promoter derived from Rous sarcoma virus, the CMV promoter derived from cytomegalovirus, β-actin and other actin promoters, and the EF1α promoter derived from the cellular elongation factor 1α gene.

Also suitable as a promoter in the plasmids of the present invention is a promoter that allows for external control over the regulation of gene expression. One way to regulate the amount and the timing of gene expression is to use an inducible promoter. Unlike a constitutive promoter, an inducible promoter is not always optimally active. An inducible promoter is capable of directly or indirectly activating transcription of one or more DNA sequences or genes in response to an inducer. Some inducible promoters are activated by physical means such as the heat shock promoter (“Hsp”). Others are activated by a chemical, for example, IPTG or tetracycline (“Tet on” system). Other examples of inducible promoters include the metallothionine promoter, which is activated by heavy metal ions, and hormone-responsive promoters, which are activated by treatment of certain hormones. In the absence of an inducer, the nucleic acid sequences or genes under the control of the inducible promoter will not be transcribed or will only be minimally transcribed. When any plasmids of the present invention contain an inducible promoter, the method of the present invention further includes the step of adding an appropriate inducing agent to the cell culture when activation of the promoter is desired. Promoters of the nucleic acid construct of the present invention may be either homologous (derived from the same species as the host cell) or heterologous (derived from a different species than the host cell.

A nucleic acid molecule of the present invention, a promoter molecule of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety, and U.S. Pat. No. 4,237,224 to Cohen and Boyer, which is hereby incorporated by reference in its entirety, which describes the production of expression systems in the form of recombinant plasmids using restriction enzyme cleavage and ligation with DNA ligase. The transcriptional and translational elements are operably linked to the nucleic acid molecule of the present invention or a fragment thereof, meaning that the resulting vector expresses the IWU-1 protein when placed in a suitable host cell. These recombinant plasmids are then introduced by means of transformation and replicated in unicellular cultures including prokaryotic organisms and eukaryotic cells grown in tissue culture.

In one aspect of the present invention, the IWU-1 nucleic acid molecule is inserted into the expression system or vector in proper sense (i.e., 5′→3′) orientation and correct reading frame. In another aspect, the IWU-1 nucleic acid molecule is inserted into the expression system or vector in the antisense (i.e., 3′→5′) orientation. The antisense form of the nucleic acid molecule is complementary to the IWU-1 nucleic acid molecule of the present invention, or complementary to a fragment of the IWU-1 nucleic acid molecule. The use of antisense RNA to down-regulate the expression of specific genes is well known (van der Krol et al., Nature, 333:866-869 (1988) and Smith et al., Nature, 334:724-726 (1988), which are hereby incorporated by reference in their entirety). Antisense nucleic acids are DNA or RNA molecules that are complementary to at least a portion of a specific mRNA molecule (Weintraub, “Antisense RNA and DNA,” Scientific American 262:40 (1990), which is hereby incorporated by reference in its entirety). Antisense methodology takes advantage of the fact that nucleic acids tend to pair with “complementary” sequences. By complementary, it is meant that polynucleotides are capable of base-pairing according to the standard Watson-Crick rules. In the target cell, the antisense nucleic acids hybridize to a target nucleic acid and interfere with transcription, and/or RNA processing, transport, translation, and/or stability. The overall effect of such interference with the target nucleic acid function is the disruption of protein expression. Accordingly, both antisense and sense forms of the nucleic acids of the present invention are suitable for use in the nucleic acid constructs of the present invention.

Once the isolated nucleic acid molecule encoding IWU-1 protein or polypeptide of the present invention or a modified version thereof has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, insect, and mammalian cells, including human. Recombinant molecules can be introduced into cells via transformation, particularly transduction, conjugation, mobilization, or electroporation. The nucleic acid sequences are cloned into the host cell using standard cloning procedures known in the art, as described by Sambrook et al., Molecular Cloning: a Laboratory Manual, Third Edition, Cold Springs Laboratory, Cold Springs Harbor, New York (2000), which is hereby incorporated by reference in its entirety.

Accordingly, another aspect of the present invention relates to a method of making a recombinant cell. Basically, this method is carried out by transforming a host cell with a nucleic acid construct of the present invention under conditions effective to yield transcription of the nucleic acid molecule in the host cell. Preferably, the nucleic acid construct of the present invention is stably inserted into the genome of the recombinant host cell as a result of the transformation.

One approach to transforming cells with a nucleic acid construct of the present invention is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford, et al., which are hereby incorporated by reference in its entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous nucleic acid molecule. Alternatively, the target cell can be surrounded by the vector so that the vector is carried into the cell by the wake of the particle. Biologically active particles (e.g., dried bacterial cells containing the vector and heterologous nucleic acid molecule) can also be propelled into plant cells. Other variations of particle bombardment, now known or hereafter developed, can also be used.

Transient expression in protoplasts allows quantitative studies of gene expression since the population of cells is very high (on the order of 10⁶). To deliver DNA inside protoplasts, several methodologies have been proposed, but the most common are electroporation (Neumann et al., “Gene Transfer into Mouse Lyoma Cells by Electroporation in High Electric Fields,” EMBO J. 1: 841-45 (1982); Wong et al., “Electric Field Mediated Gene Transfer,” Biochem Biophys Res Commun 30;107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81: 7161-65 (1984, which are hereby incorporated by reference in their entirety) and polyethylene glycol (PEG) mediated DNA uptake, Sambrook et al., Molecular Cloning: A Laboratory Manual, Chap. 16, 2d Edition, Cold Springs Laboratory, Cold Springs Harbor, New York (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment. Another appropriate method of introducing the gene construct of the present invention into a host cell is fusion of protoplasts with other entities, either minicells, cells, lysosomes, or other fusible lipid-surfaced bodies that contain the chimeric gene (Fraley, et al., Proc. Natl. Acad. Sci. USA, 79:1859-63 (1982), which is hereby incorporated by reference in its entirety).

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, New York (1989), which is hereby incorporated by reference in its entirety in its entirety.

In one aspect of the present invention, a host cell transformed with the nucleic acid construct of the present invention having the IWU-1-encoding nucleic acid molecule of the present invention will express the IWU-1 protein or polypeptide of the present invention and will exhibit an ion channel current.

The present invention also relates to a transgenic animal where the animal has an altered expression of IWU-1. This aspect involves applying the molecular biology techniques described above to prepare a nucleic acid construct having a nucleic acid molecule of the present invention incorporated in such a way as to alter the expression of the nucleic acid molecule in an animal transformed with the nucleic acid molecule, and inserting it into an appropriate expression vector for transformation into an appropriate animal. In this context, “altered” refers to either up-regulation or down-regulation of the expression an IWU-1 protein or polypeptide in a subject.

Regulation of the expression of the nucleic acid molecule of the present invention involves transformation of a cell or tissue of choice, either in vivo or ex vivo with a suitable nucleic acid construct of the present invention. Suitable constructs include, but are not limited to, constructs having one or more nucleic acid molecules of the present invention in the sense orientation, where transformation into a cell would provide an up-regulation (i.e., an increase) in the expression of IWU-1 in the transformed cell or tissue compared to an untransformed cell or tissue of the same type; constructs having an antisense nucleic acid molecule, as described above, where transformation into a cell or tissue would result in a down-regulation (i.e., decrease) in expression of IWU-1 in the transformed cell or tissue compared to an untransformed cell or tissue of the same type; or a nucleic acid construct having an untranslatable nucleic acid molecule of the present invention, prepared as described herein, supra. In one aspect of the present invention, such a transformed cell by be introduced into a subject to regulate IWU-1 expression. Also suitable is down-regulation using the gene-silencing technique known as RNA-interference (“RNAi”), as describe herein, infra.

Methods of altering the expression of endogenous proteins by transfer of recombinant genes into cell in culture and into live animals to produce transgenic animals harboring the desired gene have been developed. These include those methods include, for example, the use of retroviral vectors to introduce DNA molecules into the genome of animals (Jaenisch et al., Cell 24, 519 (1981); Soriano et al., Science 234, 1409-1413 (1986); and Stewart et al., EMBO J. 6, 383-388 (1987), which are hereby incorporated by reference in their entirety), and those methods described herein, supra. Recombinant genes have been introduced into primary cultures of bone marrow, skin, fibroblasts, hepatic or pancreatic cells and then transplanted into live animals. Transgenic animals have also been developed as bioreactors for desired biologically active molecules (U.S. Pat. No. 6,339,183 to Sun; U.S. Pat. No. 6,255,554 to Lubon et al., which are hereby incorporated by reference in their entirety).

Animals suitable for this aspect of the present invention include, but are not limited to, mammals, including humans.

Another aspect of the present invention is an isolated antibody or binding portion thereof which recognizes an IWU-1 protein or polypeptide, including, but not limited to, the protein of the present invention having an amino acid sequence of SEQ ID NO: 2 or an amino acid motif corresponding to DXFXYDXXSLRXXG. Antibodies of the present invention include those which are capable of inhibiting the activity of a polypeptide or protein of the present invention. The disclosed antibodies may be monoclonal or polyclonal. Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature, 256:495 (1975), which is hereby incorporated by reference in its entirety.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol., 6:511 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., Editors, Antibodies: a Laboratory Manual (1988), which is hereby incorporated by reference in its entirety.

The antibody, or binding portion thereof, or probe may be bound to a label effective to permit detection of the cells or tissues upon binding of the antibody, or binding portion thereof, or probe to cells or tissues contained in the sample. Examples of labels useful for diagnostic imaging, assay systems, or treatment in accordance with the present invention are radiolabels such as ¹³¹I, ¹³¹In, ¹²³I, ⁹⁹mTc, ³²O, ¹²⁵I, ³H, ¹⁴C, and ¹⁸⁸Rh, fluorescent labels such as fluorescein and rhodamine, nuclear magnetic resonance active labels, chemiluminescers such as luciferin, chromophores, and enzymatic markers such as peroxidase or phosphatase. The antibody or binding portion thereof or probe can be labeled with such reagents using techniques known in the art. For example, see Wensel and Meares, Radioimmunoimaging and Radioimmunotherapy, Elsevier, New York (1983), which is hereby incorporated by reference in its entirety, for techniques relating to the radiolabeling of antibodies. See also, D. Colcher et al., “Use of Monoclonal Antibodies as Radiopharmaceuticals for the Localization of Human Carcinoma Xenografts in Athymic Mice”, Meth. Enzymol. 121: 802-816 (1986), which is hereby incorporated by reference in its entirety.

In all aspects of the present invention involving antibodies, those antibodies may be monoclonal or polyclonal antibodies. In addition, antibody fragments, half-antibodies, hybrid derivatives, probes, and other molecular constructs may be utilized.

In addition to utilizing whole antibodies, the processes of the present invention encompass use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983), which is hereby incorporated by reference in its entirety.

In one aspect of the present invention, the antigen against which the antibody of the present invention is made against a protein or polypeptide encoded by a nucleic acid molecule that is an antisense molecule to the nucleic acid molecule of the present invention. Such an antisense nucleic acid molecule is complementary to the nucleic acid molecule of the present invention, and therefore, encodes a protein which is complementary to the protein or polypeptide of the present invention. The making of such an antisense nucleic acid molecule is carried out as described above, inserted into a nucleic acid construct and expression vector as described above or using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety. The protein or polypeptide is expressed in a recombinant system, and can be isolated and purified as described above, and used as antigen to prepare the antibody of the present invention, using the methods describe herein for antibody production.

Another aspect of the present invention is a composition having a pharmaceutical carrier and an antibody; where the antibody is made against the IWU-1 protein of polypeptide of the present invention. Such a composition may also include a component which destroys cancer cells, such as a cytotoxin. Suitable cytotoxins include, without limitation, ricin and radioactive isotopes.

Another aspect of the present invention is a method of detecting the expression of IWU-1 in a biological sample. This method involves providing an antibody or binding portion thereof which recognizes the IWU-1 polypeptide or protein of the present invention; contacting the antibody or binding portion thereof with the biological sample under conditions effective to bind an antibody or binding portion thereof with any IWU-1 protein or polypeptide present in the sample; and detecting any binding that occurs between the antibody or binding portion thereof and the biological sample, thereby indicating the expression of IWU-1 in the sample. In this aspect and all aspects of the present invention in which antibodies are used for detection, suitable antibodies include, without limitation, monoclonal and polyclonal antibodies, or an antibody fragment, such as an Fab, F (ab′)₂ or an Fv fragment. Suitable antibodies may be prepared as described above, and may be labeled for detection as described above. In this aspect and all aspects of the present invention that involves a biological sample from a subject, a suitable biological sample includes, without limitation, a body fluid, a cell, and a tissue. Any assay system capable of detecting a complex of the antigen bound to an antibody which recognizes the antigen of the present invention is suitable for this aspect, including, but not limited to, an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

The present invention also relates to a second method of detecting IWU-1 expression in a biological sample. This method involves providing a nucleic acid molecule according to claim 1 as a probe or primer in a nucleic acid hybridization assay; contacting the sample with the probe or primer under conditions effective to permit formation of a complex of the probe or primer and any nucleic acid which hybridizes to the probe or primer; and detecting formation of the complex in the sample, thereby indicating a presence of IWU-1 expression in the sample. In this aspect the nucleic acid molecule is selected from the group consisting of oligonucleotide sequences, complementary DNA and RNA, and peptide nucleic acids. Hybridization is carried out according to method described in the art, such by Sambrook et al., Molecular Cloning: A Laboratory Manual Third Edition, Cold Spring Harbor: Cold Spring Harbor Laboratory Press, New York (2001), which is hereby incorporated by reference in its entirety. Detection in this aspect is carried out by methods including, but not limited to, Northern blot, Southern blot, PCR, in-situ hybridization, or in-situ PCR.

The present invention also relates to a third method of detecting IWU-1 expression in a biological sample. This method involves providing a nucleic acid molecule of the present invention encoding an IWU-1 protein or polypeptide as a probe or primer in a gene amplification detection procedure; contacting the sample with the probe under conditions effective to amplify probe or primer-specific nucleic acid molecules; and detecting any amplified probe or primer-specific molecules, thereby indicating a presence of IWU-1 in the sample. Suitable methods for the amplification of the nucleic acid molecule encoding the protein of the present invention include, but are not limited to, polymerase chain reaction (PCR), ligase chain reaction (LCR) (Barany, Proc. Nat'l Acad. Sci. U.S.A., 88, 189 (1991), which is hereby incorporated by reference in its entirety), ligase detection reaction (LDR); LDR-PCR; strand displacement amplification (Walker et al., Nucleic Acids Res, 20, 1691 (1992); Walker et al., Proc. Nat'l Acad. Sci. U.S.A., 89, 392 (1992), which are hereby incorporated by reference in their entirety); transcription-based amplification (Kwoh et al., Proc. Nat'l Acad. Sci. U.S.A., 86, 1173 (1989), which is hereby incorporated by reference in its entirety); self-sustained sequence replication (or “3SR”) (Guatelli et al., Proc. Nat'l Acad. Sci. U.S.A., 87, 1874 (1990), which is hereby incorporated by reference in its entirety); nucleic acid transcription-based amplification System (“TAS”); the Q-beta replicase system (Lizardi et al., Biotechnology, 6, 1197 (1988), which is hereby incorporated by reference in its entirety); hybridization signal amplification (HSAM); nucleic acid sequence-based amplification (NASBA) (Lewis, R., Genetic Engineering News, 12(9), 1 (1992), which is hereby incorporated by reference in its entirety), the repair chain reaction (RCR) (Lewis, R., Genetic Engineering News, 12(9), 1 (1992), which is hereby incorporated by reference), and boomerang DNA amplification (BDA) (Lewis, R., Genetic Engineering News, 12(9), 1 (1992), which is hereby incorporated by reference in its entirety); or branched-DNA methods.

In general, amplification techniques such as the foregoing involve the use of a probe, a pair of probes, or two pairs of probes which specifically bind to the nucleic acid molecule of interest, but do not bind to other nucleic acid molecules, under the same hybridization conditions, and which serve as the primer or primers for the amplification reaction.

The method described above may also include the steps of measuring mRNA levels by quantitating the levels of antisense RNA transcripts for control genes and then comparing the mRNA levels for the transcripts to the mRNA levels for the control gene transcripts using multivariate analysis. The use of control levels allows for the rapid identification of mRNA transcripts which are aberrantly expressed in the diseased cells. Detection may be carried out using any methods commonly associated with the method of amplification selected by the user, including, but not limited to, gel electrophoresis, array-capture, and direct sequencing. The nucleic acid probes in this aspect of the present invention can be labeled or tagged in accordance with the detection method of choice.

Another aspect of the present invention is a method of treating a disease condition in a subject. This method involves providing a nucleic acid molecule encoding an IWU-1 polypeptide or protein or probe thereto; contacting the nucleic acid molecule encoding an IWU-1 polypeptide or protein or probe thereto with a cell or tissue sample of a subject under conditions effective to bind to cells overexpressing IWU-1 from the cell or tissue sample; and removing cells or tissues which are selected by the nucleic acid molecule or probe thereto. “Overexpressing” as used herein refers to the excessive expression of a gene product. Many disease conditions, particularly cancers, have been associated with the overexpression of gene products. The overexpression of IWU-1 may be associated with one or more disease conditions. Thus, this method is suitable for the treatment of cancerous conditions, including tumors of various kinds, as well as hematopoietic cancers such as leukemia, and other diseases, including, but not limited to hypertension, hypotension, ischemia, inflammation, arthritis, diabetic retinopathy, myocardial infarction, and cardio-vascular disease.

The method involves bringing a biological sample containing disease cells into contact with the nucleic acid of the present invention under conditions to allow the convenient selection and removal of the diseases cells from the biological sample. This method is suitable for all types of cells and tissues, including, without limitation, bone marrow, blood, breast tissue or tissue from organs. In this and all aspects of the present invention that involve treatment of a disease condition in a subject, suitable subjects include, but are not limited to, mammals, including humans.

The present invention also relates to another method of treating a disease condition in a subject. This method involves providing an antibody or binding portion thereof that recognizes an IWU-1 polypeptide or protein; contacting the antibody or binding portion thereof that recognizes the IWU-1 polypeptide or protein with a cell or tissue sample of the subject under conditions effective to bind to cells overexpressing IWU-1 from the cell or tissue sample; and removing cells or tissues which bind to the an antibody or binding portion thereof, thereby treating the disease. Suitable antibodies, the making, labeling, and detecting thereof are as described above. Suitable cells and tissues, suitable subjects, and disease conditions to which this aspect applies are all as described above.

The present invention relates to a third method of treating a disease condition in a subject. This method involves providing a therapeutic amount of a pharmaceutical conjugate having an antibody or binding portion thereof against an IWU-1 protein or polypeptide and a cytotoxic component; and administering the conjugate to the subject under conditions effective to form an immune complex with an IWU-1 protein polypeptide or protein. In this aspect of the present invention administering is carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane. Suitable cytotoxic components for the conjugate of the present invention and the disease conditions to which this aspect applies are as described above.

Another aspect of the present invention is a vaccine having an isolated IWU-1 polypeptide or protein according to the present invention or an antigenic fragment thereof.

Another aspect of the present invention is a fourth method of treating a disease condition in a subject. This method involves providing a composition having an IWU-1 polypeptide or protein of the present invention or an antigenic fragment thereof; and administering a therapeutically effective amount of the composition to a subject.

The present invention also relates to a method of regulating IWU-1 expression in a subject. Regulation may include up- or down-regulation of IWU-1 expression. In the aspect of the present invention involving up-regulation, this method involves administering to a subject the nucleic acid molecule of the present invention described herein, supra. This may be in the form of a nucleic acid construct having one or more IWU-1 nucleic acid molecules of the present invention inserted. In another aspect, in which down regulation of expression is desired, this method involves administering to a subject the antisense nucleic acid molecule of the present invention described herein, supra. Alternatively, the nucleic acid construct of the present invention may be configured so that the nucleic acid molecule encodes an mRNA which is not translatable, i.e., does not result in the production of a protein or polypeptide. This is achieved, for example, by introducing into the desired nucleic acid sequence of the present invention one or more premature stop codons, adding one or more bases (except multiples of 3 bases) to displace the reading frame, and removing the translation initiation codon (U.S. Pat. No. 5,583,021 to Dougherty et al., which is hereby incorporated by reference in its entirety). This can involve the use of a primer to which a stop codon, such as TAATGA, is inserted into the sense (or “forward”) PCR-primer for amplification of the full nucleic acid, between the 5′ end of that primer, which corresponds to the appropriate restriction enzyme site of the vector into which the nucleic acid is to be inserted, and the 3′ end of the primer, which corresponds to the 5′ sequence of the enzyme-encoding nucleic acid.

Genes can be effective as silencers in the non-translatable antisense forms, as well as in the non-translatable sense form (Baulcombe, D. C., “Mechanisms of Pathogen-Derived Resistance to Viruses in Transgenic Plants,” Plant Cell 8:1833-44 (1996); Dougherty, W. G., et al., “Transgenes and Gene Suppression: Telling us Something New?” Current Opinion in Cell Biology 7:399-05 (1995); Lomonossoff, G. P., “Pathogen-Derived Resistance to Plant Viruses,” Ann. Rev. Phytopathol. 33:323-43 (1995), which are hereby incorporated by reference in their entirety).

In the aspect of the present invention in which down-regulation of IWU-1 expression is desired, the method may involve an RNA-based form of gene-silencing known as RNA-interference (RNAi). Numerous reports have been published on critical advances in the understanding of the biochemistry and genetics of both gene silencing and RNAi (Matzke et al., “RNA-Based Silencing Strategies in Plants,” Curr. Opin. Genet. Dev. 11(2):221-227 (2001), which is hereby incorporated by reference in its entirety). In RNAi, the introduction of double stranded RNA (dsRNA) into animal or plant cells leads to the destruction of the endogenous, homologous mRNA, phenocopying a null mutant for that specific gene. In both post-transcriptional gene silencing and RNAi, the dsRNA is processed to short interfering molecules of 21-, 22- or 23-nucleotide RNAs (siRNA) by a putative RNAaseIII-like enzyme (Tuschl T., “RNA Interference and Small Interfering RNAs,” Chembiochem 2: 239-245 (2001); Zamore et al., “RNAi: Double Stranded RNA Directs the ATP-Dependent Cleavage of mRNA at 21 to 23 Nucleotide Intervals,” Cell 101, 25-3, (2000), which are hereby incorporated by reference in their entirety). The endogenously generated siRNAs mediate and direct the specific degradation of the target mRNA. In the case of RNAi, the cleavage site in the mRNA molecule targeted for degradation is located near the center of the region covered by the siRNA (Elbashir et al., “RNA Interference is Mediated by 21- and 22-Nucleotide RNAs,” Gene Dev. 15(2):188-200 (2001), which is hereby incorporated by reference in its entirety). In one aspect, dsRNA for the nucleic acid molecule of the present invention can be generated by transcription in vivo. This involves modifying the nucleic acid molecule of the present invention for the production of dsRNA, inserting the modified nucleic acid molecule into a suitable expression vector having the appropriate 5′ and 3′ regulatory nucleotide sequences operably linked for transcription and translation, and introducing the expression vector having the modified nucleic acid molecule into a suitable host cell or subject. In another aspect of the present invention, complementary sense and antisense RNAs derived from a substantial portion of the coding region of the nucleic acid molecule of the present invention are synthesized in vitro. (Fire et al., “Specific Interference by Ingested dsRNA,” Nature 391:806-811 (1998); Montgomery et al, “RNA as a Target of Double-Stranded RNA-Mediated Genetic Interference in Caenorhabditis elegans,” Proc. Natl Acad Sci USA 95: 15502-15507; Tabara et al., “RNAi in C. elegans: Soaking in the Genome Sequence,” Science 282:430-431 (1998), which are hereby incorporated by reference in their entirety). The resulting sense and antisense RNAs are annealed in an injection buffer, and dsRNA is administered to the subject using any method of administration described herein, infra.

The present invention also relates to a method of gene therapy that involves administering to a subject the nucleic acid molecule of the present invention that encodes an IWU-1 protein or polypeptide. This method may be carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane. Exemplary delivery devices include, without limitation, liposomes, transdermal patches, implants, and syringes.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which are hereby incorporated by reference in their entirety). When liposomes are endocytosed by a target cell, for example, they can be routed to acidic endosomes which will destabilize the liposome and result in drug release.

Alternatively, the delivery vehicle includes an enzymatically stable conjugate that includes a polymer. The IWU-1 protein or polypeptide is chemically conjugated to the polymer.

The liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release. This liposome delivery system can also be made to accumulate at a target organ, tissue, or cell via active targeting. In accordance with the present invention, liposomes can be targeted to an organ, cell or tissue of choice by incorporating into the liposome bilayer a molecule which targets receptors for the organ, tissue, or cell of choice.

Other suitable protein delivery systems may be used, including, without limitation, a transdermal patch, and implantable or injectable protein depot compositions, which provide long-term delivery of fusion proteins (U.S. Pat. No. 6,331,311 to Brodbeck et al., which is hereby incorporated by reference in its entirety). Other delivery systems which are known to those of skill in the art can also be employed to achieve the desired delivery of the fusion protein to smooth muscle cells in vivo to effect this aspect of the present invention.

Alternatively, the gene therapy aspect of the present invention can be carried out by transforming a suitable host cell with an infective transformation vector harboring the nucleic acid encoding the IWU-1 protein or polypeptide. Exemplary infective transformation vectors include, without limitation, an adenovirus vector, a retrovirus vector, or a lentivirus vector harboring the nucleic acid encoding the retinoid-inducible protein or polypeptide. Such vectors, prepared as described above with suitable transcriptional and translational regulatory elements, are capable of expressing the IWU-1 protein or polypeptide protein or polypeptide in a transformed cell. The introduction of a IWU-1 protein or polypeptide of the present invention may be carried out by employing a delivery vehicle having the IWU-1 protein or polypeptide. Exemplary delivery vehicles for this aspect of the present invention include, without limitation, a fusion protein having a IWU-1 protein or polypeptide of choice and a ligand domain recognized by the cell of choice; a liposome vehicle, in its various forms as described above and known in the art; or an enzymatically stable conjugate having a polymer and IWU-1 protein or polypeptide conjugated to the polymer. Other delivery vehicles known to those in the art are also suitable, including a catheter device to deploy the delivery vehicles as described above. In this aspect of the present invention, suitable subjects include, without limitation, mammals, including humans.

Another aspect of the present invention is a method of screening drugs that regulate IWU-1 activity. This involves providing the IWU-1 protein or polypeptide of the present invention; providing a reagent upon which IWU-1 exerts activity; providing a test compound; combining the IWU-1 protein or polypeptide, the reagent, and the test compound in a mixture; determining the activity of IWU-1 upon the reagent in the mixture; and measuring any difference between the activity of IWU-1 upon the reagent with and without the test compound.

Another aspect of the present invention is a method of screening drugs that regulate IWU-1 expression. This method involves transforming a host cell with a nucleic acid molecule encoding an IWU-1 protein or polypeptide; culturing the transformed cells; adding a test compound to the culture containing the transformed cells; and determining whether the test compound regulates expression of IWU-1 in the transformed cells. Transformation according to this aspect of the present invention may be carried out as described herein, supra, or as described in the art, such as by Sambrook et al., Molecular Cloning: A Laboratory Manual, Third Edition, Cold Spring Harbor: Cold Spring Harbor Laboratory Press, New York (2000), which is hereby incorporated by reference in its entirety, and U.S. Pat. No. 4,237,224 to Cohen and Boyer, which is hereby incorporated by reference in its entirety. Suitable host cells in this aspect of the present invention include bacterial cells, a virus, yeast, insect cells, and mammalian cells, including, but not limited to, human cells. In one embodiment of this aspect of the present invention, cells are isolate from a transgenic animal having an altered expression of IWU-1, such as described above. A test compound is added to the isolated cells; and it is determined whether the test compound regulates the expression of IWU-1 in the isolated cells. Exemplary methods for the determination of IWU-1 expression include, without limitation, Northern, Southern, and Western blotting, and in situ hybridization.

The present invention also relates to another method of screening drugs that regulate IWU-1 expression. This method involves isolating cells from a transgenic animal having an altered expression of IWU-1, where the altered expression is the result of the introduction of a nucleic acid molecule according to the present invention into the animal; adding a test compound to the isolated cells; and determining whether the test compound regulates the expression of IWU-1 in the isolated cells. The transgenic animal from which the cells are isolated is any of the transgenic animals described herein, supra, having an altered expression of IWU-1. Suitable hosts and exemplary methods of determination of IWU-1 expression are as described just above.

### EXAMPLES

### Example 1

### Identification of ATP1γ1/PLM/MAT8-type Gene from Human Long-Term Bone Marrow Culture

Using an RNA fingerprinting method to analyze the gene expression profile of a three-dimensional human long-term bone marrow culture (LTBMC) (Wang et al., “Multilineal Hemopoiesis in a Three-Dimensional Murine Long-Term Bone Marrow Culture,” Exp. Hematol. 23:26-32 (1995); Mantalaris et al., “Engineering a Human Bone Marrow Model: a Case Study on Ex Vivo Erythropoiesis,” Biotechnol. Prog. 14:126-133 (1998), which are hereby incorporated by reference in their entirety), a novel gene was identified with high homology to the ATP1γ1/PLM/MAT8 family proteins in the region containing the putative transmembrane domain. The gene expression profile of the LTBMC was analyzed using the RNA arbitrarily primed (RAP)-PCR technique (Welsh et al., “Arbitrarily Primed PCR Fingerprinting of RNA,” Nucleic Acids Res. 20:4965-4970 (1992), which is hereby incorporated by reference in its entirety) as a fingerprinting method. In this method, mRNA was isolated from the adherent cell population, obtained by trypsinization of the three-dimensional human bone marrow culture at week four. cDNA was then prepared and subsequently amplified using the 18-base arbitrary primer (SEQ ID NO: 12): AATCTAGAGCTCCAGCAG. The RAP-PCR products were separated by gel electrophoresis and the DNA was visualized by silver staining (Lohmann et al., “REN Display, A Rapid and Efficient Method for Nonradioactive Differential Display and mRNA Isolation,” BioTechniques 18:201-202 (1995), which is hereby incorporated by reference in its entirety). A 270 bp cDNA fragment (band 6-6) was eluted, reamplified and cloned into the pCR 2.1-TOPO TA cloning vector (Invitrogen, Carlsbad, Calif.) and its nucleotide sequence determined.

### Example 2

### Analysis of Putative IWU-1 Nucleic Acid Molecule

Homology search using the BLAST program (Altschul et al., “Gapped BLAST and PSI-BLAST: a New Generation of Protein Database Search Programs,” Nucleic Acids Res. 25:3389-3402 (1997), which is hereby incorporated by reference in its entirety) revealed that the sequence of band 6-6 matches that of Unigene Hs. 25334. One of the Hs. 25334 Unigene clones, the EST clone AI358167, was obtained and its entire nucleotide sequence determined. The nucleotide sequence of AI358167 (SEQ ID NO: 15) encompassed the entire sequence of band 6-6 with both 5′ and 3′ extensions, seen in FIG. 1. The 647 bp nucleotide sequence of AI358167 (SEQ ID NO: 15) contained a putative open reading frame with a poly(A) tail. Also shown in FIG. 1 is the open reading frame, presumed to start from the first translation start codon, ATG, at positions 19-21, and run to a stop codon, GTG, at position 363-365, designated SEQ ID NO: 1, herein. It encoded 115 amino acids (SEQ ID NO: 2) with a calculated molecular mass of 12.9 kDa. The open reading frame is flanked by in-frame stop codons, indicating the clone contains the entire coding region of the protein. The translated protein was name IWU-1.

BLAST homology search indicated that the IWU-1 protein contained an ATP1γ1/PLM/MAT8 family consensus sequence (positions 70-83 of its amino acid sequence)(SEQ ID NO: 16) and exhibited high homology to the proteins of the family in the region containing the putative transmembrane domain, as shown in FIG. 2. Within this region, IWU-1 exhibited 68% homology (48% identity) to human ATP1γ1 (Kim et al., “Cloning and Expression of Human cDNA Encoding Na⁺, K⁺-ATPase γ-subunit,” Biochim. Biophys. Acta 1350:133-135 (1997), which is hereby incorporated by reference in its entirety), 68% homology (51% identity) to human MAT-8 (Morrison et al., “Mat-8, a Novel Phospholemman-like Protein Expressed in Human Breast Tumors, Induces a Chloride Conductance in Xenopus Oocytes,” J. Biol. Chem. 270:2176-2182 (1995), which is hereby incorporated by reference in its entirety), 74% homology (51% identity) to human PLM (Chen et al., “Characterization of the Human and Rat Phospholemman (PLM) cDNA and Localization of the Human PLM Gene to Chromosome 19q13.1,” Genomics 41:435-443 (1995), which is hereby incorporated by reference in its entirety), and 64% homology (41% identity to rat CHIF (Attali et al., “A Corticosteroid-Induced Gene Expressing an ‘Isk-like’ K⁺ Channel Activity in Xenopus Oocytes,” Proc. Natl. Acad. Sci. USA 92:6092-60956 (1995), which is hereby incorporated by reference in its entirety). It is noteworthy that, in this region, IWU-1 exhibited 97% homology (91% identity) to mouse RIC (originally ET-8) (Fu et al., “E2a-Pbx1 Induces Aberrant Expression of Tissue-Specific and Developmentally Regulated Genes When Expressed in NIH 3T3 Fibroblasts,” Mol. Cell. Biol. 17:1503-1512 (1997), which is hereby incorporated by reference in its entirety). In addition, in the putative cytoplasmic region at the C-terminal side, IWU-1 (positions 106-115), showed 60% homology (50% identity) to mouse RIC (positions 169-178). However, IWU-1 showed no significant homology to the putative extracellular region of the mouse RIC protein (positions 1-132), including the proline rich region and the putative signal peptide.

### Example 3

### Homology Analysis of N-Terminal Region of IWU-1

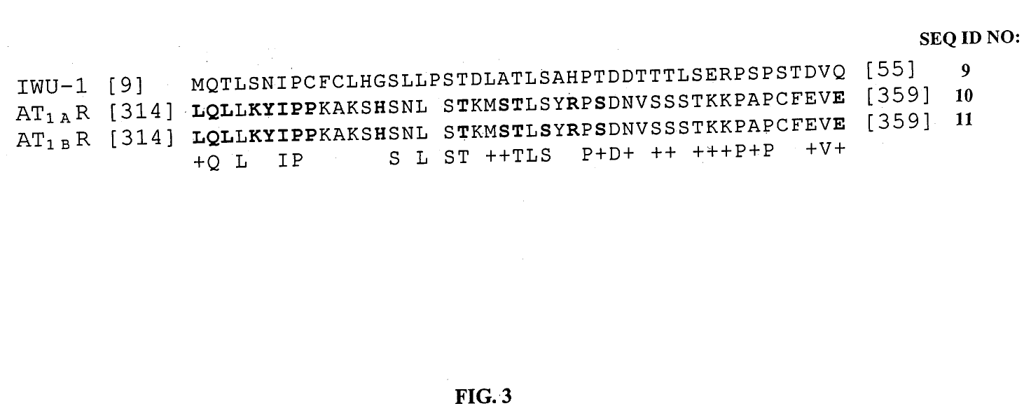

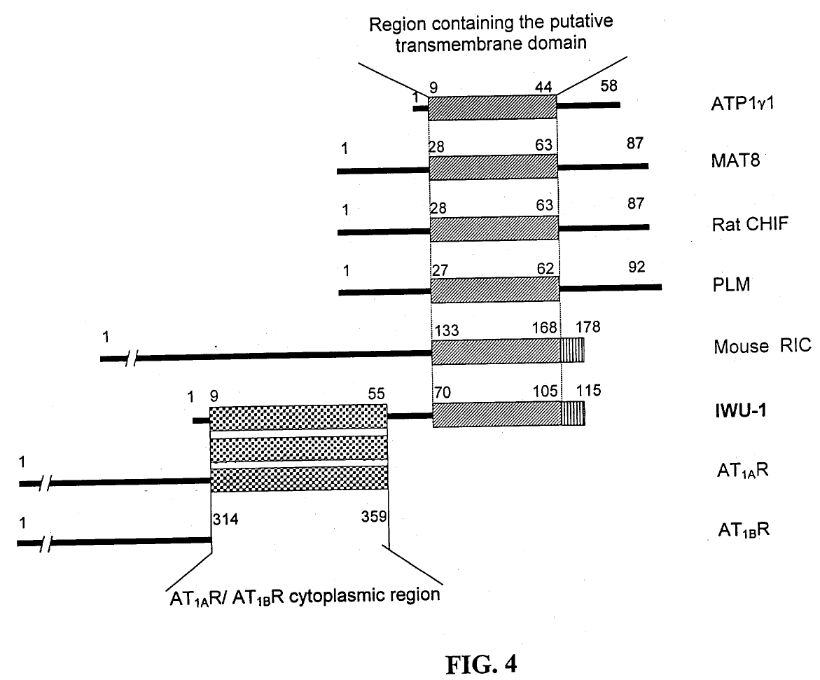

The N-terminal region of IWU-1 was also analyzed by the BLAST program. As shown in FIG. 3, IWU-1 (positions 9-55) exhibited 61% homology (34% identity) to the cytoplasmic region of the human type 1A (At_{1A}R) (Takayanagi et al., “Molecular Cloning, Sequence Analysis and Expression of a CDNA Encoding Human Type-1 Angiotensin 11 Receptor,” Biochem. Biophys. Res. Commun. 183:910-916 (1992); Mauzy et al., “Cloning, Expression, and Characterization of a Gene Encoding the Human Angiotensin II Type 1A Receptor,” Biochem. Biophys. Res. Commun. 186:277-284 (1992), which are hereby incorporated by reference in their entirety) and the type 1B (AT_{1B}R) angiotensin II receptors (Konishi et al., “Novel Subtype of Human Angiotensin II Type 1 Receptor: cDNA Cloning and Expression,” Biochem. Biophys. Res. Commun. 199:467-474 (1994), which is hereby incorporated by reference in its entirety). This region is completely conserved between AT_{1A}R and AT_{1B}R, which are G-protein coupled receptors each with seven transmembrane segments. The sequence is on the cytoplasmic side for both proteins and is located at their C-termini. It contains several potential phosphorylation sites for protein kinase C ([ST]=x=[RK]) (Konishi et al., “Novel Subtype of Human Angiotensin II Type 1 Receptor: cDNA Cloning and Expression,” Biochem. Biophys. Res. Commun. 199:467-474 (1994); Kishimoto et al., “Studies on the Phosphorylation of Myelin Basic Protein by Protein Kinase C and Adenosine 3′:5′-Monophosphate-Dependent Protein Kinase,” J. Biol. Chem. 260:12492-12499 (1985)): PRO-SITE accession No. PS00005). One of these sites appears to be conserved in IWU-1 (Ser 45; FIG. 3). In addition, Thr 60 (FIG. 1) is another potential protein kinase C phosphorylation site outside the transmembrane domain. Compared to the ATP1γ1/PLM/MAT8 family proteins, IWU-1 has an extended N-terminal region outside the putative transmembrane domain, as seen in FIG. 4. It is longer than those of the other proteins except mouse RIC. This region may, therefore, play an important role for the function of IWU-1. The sequence homology among IWU-1, the ATP1γ1/PLM/MAT8 family proteins, and AT_{1A}R/AT_{1B}R, is summarized in FIG. 4.

It has been reported that many proteins of the ATP1γ1/PLM/MAT8 family, including human and rat PLM, and human MAT-8 (Chen et al., “Characterization of the Human and Rat Phospholemman (PLM) cDNA and Localization of the Human PLM Gene to Chromosome 19q13.1,” Genomics 41:435-443 (1997), which is hereby incorporated by reference in its entirety) and rat CHIF (Attali et al., “A Corticosteroid-Induced Gene Expressing an ‘Isk-like’ K⁺ Channel Activity in Xenopus Oocytes,” Proc. Natl. Acad. Sci. USA 92:6092-60956 (1997), which is hereby incorporated by reference in its entirety), when expressed in Xenopus oocyte, induce Cl⁺ or K⁺ channel activities. ATP1γ1 (Na⁺, K⁺-ATPaseγ-subunit) is involved in active transport of Na⁺ and K⁺ ions across the membrane. It has also been implicated in controlling and maintaining kidney functions and tumorigenesis in kidney (Kim et al., “Cloning and Expression of Human cDNA Encoding Na⁺, K⁺-ATPaseγ-Subunit,” Biochim. Biophys. Acta 1350:133-135 (1997), which is hereby incorporated by reference in its entirety). These observations suggest that IWU-1 serves as an ion channel protein or as an ion channel regulator, and as such, may have a role in disease conditions including hypertension, hypotension, ischemia, diabetic retinopathy, and cardio-vascular disease. Another member of this gene family with which IWU-1 shares homology (see FIG. 2 and FIG. 4) is RIC, also known as EF-8. RIC has been shown to be strongly up-regulated by E2a-Pbx1 oncogene and moderately up-regulated by oncogenic versions of Lck, Ras, Neu, Src, and Abl ((Fu et al., 3-15 “E2a-Pbx1 Induces Aberrant Expression of Tissue-Specific and Developmentally Regulated Genes When Expressed in NIH 3T3 Fibroblasts,” Mol Cell Biol 17: 1503-1512 (1997), which is hereby incorporated by reference in its entirety).

The homology to angiotensin receptors AT_{1A}R and AT_{1B}R, as described in Example 3, supra, suggests a role for IWU-1 in a variety of physiological processes and disease conditions. The rennin-angiotensin system (RAS) is involved in a complex mechanism that serves to preserve the blood supply to organs for maintenance of cellular function. Many tumors, both benign and malignant, express rennin and angiotensin (Achard et al., “Protection Against Ischemia: A Physiological Function of the Renin-Angiotensin System,” Biochemical Pharmacology 62(3):261-271 (2001), which is hereby incorporated by reference in its entirety). Recent research indicates that RAS, directly or indirectly, is involved in situations in which the restoration of blood supply is critical for the viability of cells. Thus, angiotensin is considered likely to be involved in other conditions, such as inflammation, arthritis, diabetic retinopathy, and retrolental fibroplasias (Achard et al., “Protection Against Ischemia: A Physiological Function of the Renin-Angiotensin System,” Biochemical Pharmacology 62(3):261-271 (2001), which is hereby incorporated by reference in its entirety). Angiotensin has also been implicated in the progression of heart failure (McMurray et al., “Angiotensin-(1-7) Attenuates the Development of Heart Failure After Myocardial Infarction in Rats,” Circulation 106(20):el47(2002), which is hereby incorporated by reference in its entirety); hypertension (Sica, D. A., “Pharmacology and Clinical Efficacy of Angiotensin Receptor Blockers,” American J. Hypertension 14:242S-247S (2001), which is hereby incorporated by reference in its entirety); hypotension (Pastor et al., “Treatment of Hypotension in Septic Shock,” Lancet 347(9001):622-623 (1996); and proliferation and chemotaxis of multiple cell-types, including hematopoietic progenitors and mesenchymal cells (Rodgers et al., “Accelerated Recovery From Irradiation Injury By Angiotensin Peptides,” Cancer Chemotherapy & Pharmacology 49(5): 403-11 (2002), which is hereby incorporated by reference in its entirety).

### Example 4

### IWU-1 Gene Expressed in Human Bone Marrow

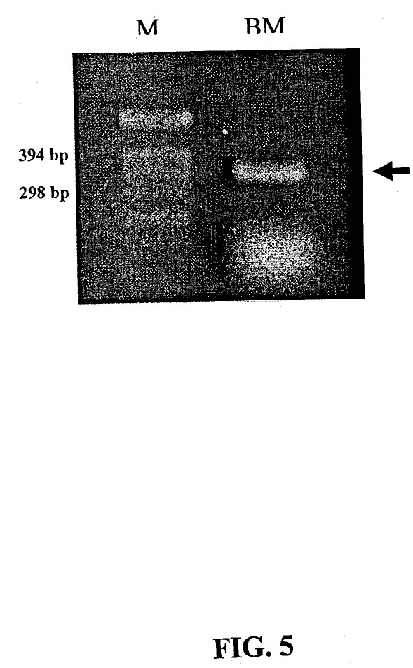

The IWU-1 gene was identified from the human LTBMC as described above. To determine if the gene is expressed in human bone marrow, PCR was performed on a human bone marrow cDNA library constructed using pooled fresh bone marrow samples from 24 individuals (Clontech Laboratories, Inc., Palo Alto, Calif.). Two IWU-1 specific sequences, (SEQ ID NO: 13) (forward primer) TCTCAGTAATATCCCTTGTTTCTGC and (SEQ ID NO: 14) (reverse primer), GTTGTCAGCTCCTGTTTCTGATG, corresponding to nucleotides 51-75 and 370-392 respectively, as shown in FIG. 1, were used as PCR primers. Analysis of the PCR product by gel electrophoresis revealed a single band of approximately 340 bp, shown in FIG. 5, consistent with the expected size (342 bp). The sequence of the PCR product confirmed that the IWU-1 gene was correctly amplified. The IBU-1 gene is therefore expressed in human bone marrow as well as the bone marrow culture system (Mantalaris et al., “Engineering a Human Bone Marrow Model: a Case Study on ex vivo Erythropoiesis,” Biotechnol. Pros. 14:126-133 (1998), which is hereby incorporated by reference in its entirety). In addition, the Unigene Hs. 25334, from which the IWU-1 sequence was derived, has been mapped to chromosome 19 and obtained from many cell and tissue types including T- and B-lymphocytes, adipose tissue, aorta, breast, fetal heart, lung, pancreatic islet, prostate, and pregnant uterus. It has also been cloned from many types of tumors and leukemia. The gene thus appears to be expressed in a large variety of cell and tissue types.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An isolated nucleic acid molecule encoding an IWU-1 protein or polypeptide, wherein the nucleic acid molecule either: 1) has a nucleotide sequence of SEQ ID NO: 1; 2) encodes a protein or polypeptide having an amino acid sequence of SEQ ID NO: 2; 3) has a nucleotide sequence that is at least 55% similar to the nucleotide sequence of SEQ ID NO: 1 by basic BLAST using default parameters analysis, or 4) hybridizes to the nucleotide sequence of SEQ ID NO: 1 under stringent conditions characterized by a hybridization buffer comprising 5×SSC at a temperature of 55° C.

2. The isolated nucleic acid molecule according to claim 1, wherein the nucleic acid molecule encodes a protein or polypeptide having an amino acid sequence of SEQ ID NO: 2.

3. The isolated nucleic acid molecule according to claim 1, wherein the nucleic acid molecule has a nucleotide sequence of SEQ ID NO: 1.

4. The isolated nucleic acid molecule according to claim 1, wherein the nucleic acid molecule has a nucleotide sequence that is at least 55% similar to the nucleotide sequence of SEQ ID NO: 1 by basic BLAST using default parameters analysis.

5. The isolated nucleic acid molecule according to claim 1, wherein the nucleic acid molecule hybridizes to the nucleotide sequence of SEQ ID NO: 1 under stringent conditions characterized by a hybridization buffer comprising 5×SSC at a temperature of 55° C.

6. The isolated nucleic acid molecule according to claim 1, wherein the nucleic acid molecule encodes an ion channel protein or ion regulator comprising:
a transmembrane domain; and
an angiotensin-homologous domain.

7. The isolated nucleic acid molecule of claim 1, wherein the protein or polypeptide is expressed in tumor cells.

8. An expression vector comprising transcriptional and translational regulatory nucleotide sequences operably linked to a nucleic acid molecule according to claim 1.

9. An expression vector according to claim 8, wherein the nucleic acid molecule is in proper sense orientation and correct reading frame.

10. A host cell transduced with nucleic acid molecule according to claim 1.

11. A host cell according to claim 10, wherein the cell is selected from the group consisting of a bacterial cell, a virus, a yeast cell, insect cell, and a mammalian cell.

12. A cell according to claim 10, wherein the cell expresses the amino acid encoded by the nucleic acid molecule and exhibits an ion channel current.

13. An antisense nucleic acid molecule which is complementary to the nucleic acid molecule according to claim 1.

14. An expression vector comprising transcriptional and translational regulatory nucleotide sequences operably linked to the antisense nucleic acid molecule according to claim 13.

15. A host cell transduced with the antisense nucleic acid molecule according to claim 13.

16. A host cell according to claim 15, wherein the host cell is selected from the group consisting of a bacterial cell, a virus, a yeast cell, an insect cell, and a mammalian cell.

17. A double-stranded RNA molecule transcribed from the nucleic acid molecule according to claim 1.

18. An expression vector comprising transcriptional and translational regulatory nucleotide sequences operably linked to the modified nucleic acid molecule according to claim 17.

19. A host cell transduced with the modified nucleic acid molecule according to claim 17.

20. A host cell according to claim 19, wherein the host cell is selected from the group consisting of a bacterial cell, a virus, a yeast cell, an insect cell, and a mammalian cell.

21. An isolated IWU-1 protein or polypeptide, wherein the protein or polypeptide has an amino acid sequence of SEQ ID NO: 2 or comprises an amino acid motif corresponding to DXFXYDXXSLRXXG.

22. The isolated IWU-1 protein or polypeptide according to claim 21, wherein the protein or polypeptide has an amino acid sequence of SEQ ID NO: 2.

23. The isolated IWU-1 protein or polypeptide according to claim 21, wherein the protein or polypeptide comprises an amino acid motif corresponding to DXFXYDXXSLRXXG.

24. An isolated antibody, or binding portion thereof, which recognizes the protein or polypeptide according to claim 21.

25. The isolated antibody according to claim 24, wherein the antibody is a polyclonal antibody.

26. The isolated antibody according to claim 24, wherein the antibody is a monoclonal antibody.

27. A composition comprising a pharmaceutical carrier and an antibody according to claim 24.

28. A composition according to claim 27 further comprising a cytotoxin.

29. The composition according to claim 28, wherein the cytotoxin is selected from the group consisting of ricin and radioactive isotopes.

30. A method of detecting the expression of IWU-1 in a biological sample comprising:
providing an antibody or binding portion thereof according to claim 24;
contacting the antibody or binding portion thereof with the biological sample under conditions effective to bind an antibody or binding portion thereof with any IWU-1 protein or polypeptide present in the sample; and
detecting any binding that occurs between the antibody or binding portion thereof and the biological sample, thereby indicating the expression of IWU-1 in the sample.

31. The method according to claim 30, wherein the biological sample is selected from the group consisting of a body fluid, a cell, and a tissue.

32. The method according to claim 30, wherein an antibody is used to carry out the method and the antibody is selected from the group consisting of a monoclonal antibody and a polyclonal antibody.

33. The method according to claim 32, wherein the antibody is a monoclonal antibody.

34. The method according to claim 32, wherein the antibody is a polyclonal antibody.

35. The method according to claim 30, wherein a binding portion thereof is used to carry out the method and the binding portion is selected from the group consisting of an Fab fragment, an F(ab′)₂ fragment, and an Fv fragment.

36. The method according to claim 30, wherein the antibody or binding portion thereof has a label to permit detection of binding of the antibody or binding portion thereof to the biological sample.

37. The method according to claim 36, wherein the label is selected from the group consisting of a fluorescent label, a radioactive label, a nuclear magnetic resonance active label, a luminescent label, and a chromophore label.

38. The method according to claim 30, wherein said detecting is carried out using an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

39. A method of detecting IWU-1 expression in a biological sample comprising:
providing a nucleic acid molecule according to claim 1 as a probe or primer in a nucleic acid hybridization assay;
contacting the sample with the probe or primer under conditions effective to permit formation of a complex of the probe or primer and any nucleic acid molecule which hybridizes to the probe or primer; and
detecting formation of complex in the sample, thereby indicating a presence of IWU-1 expression in the sample.

40. The method according to claim 39, wherein the nucleic acid molecule provided encodes an IWU-1 protein or polypeptide, and the nucleic acid molecule either: 1) has a nucleotide sequence of SEQ ID NO: 1; 2) encodes a protein or polypeptide having an amino acid sequence of SEQ ID NO: 2; 3) has a nucleotide sequence that is at least 55% similar to the nucleotide sequence of SEQ ID NO: 1 by basic BLAST using default parameters analysis, or 4) hybridizes to the nucleotide sequence of SEQ ID NO: 1 under stringent conditions characterized by a hybridization buffer comprising 5×SSC at a temperature of 55° C.

41. The method according to claim 39, wherein the nucleic acid molecule is selected from the group consisting of oligonucleotide sequences, complementary DNA and RNA, and peptide nucleic acids.

42. The method according to claim 39, wherein said detecting is carried out by Northern blot, Southern blot, PCR, in-situ hybridization, or in-situ PCR.

43. A method of detecting IWU-1 expression in a biological sample comprising:
providing a nucleic acid molecule according to claim 1 as a probe or primer in a gene amplification detection procedure;
contacting the sample with the probe or primer under conditions effective to amplify probe or primer-specific nucleic acid molecules; and
detecting any amplified probe or primer-specific molecules, thereby indicating a presence of IWU-1 in the sample.

44. The method according to claim 43, wherein the gene amplification detection procedure is selected from the group consisting of polymerase chain reaction (PCR), ligase chain reaction (LCR), ligase detection reaction (LDR), LDR-PCR, strand displacement amplification, hybridization signal amplification (HSAM), self-sustained sequence (3SR) replication, Q-beta replicase, nucleic acid sequence based amplification (“NASBA”), transcription-based amplification System (“TAS”), and branched-DNA.

45. The method according to claim 43, wherein the detection is carried out using gel electrophoresis, array-capture, or direct sequencing.

46. A method of treating a disease condition in a subject comprising:
providing a nucleic acid molecule according to claim 1 or a probe thereto;
contacting the nucleic acid molecule or probe thereto with a cell or tissue sample of a subject under conditions effective to bind to cells overexpressing IWU-1 from the cell or tissue sample; and
removing cells or tissues which are selected by the nucleic acid molecule or probe thereto, thereby treating the disease condition.

47. The method according to claim 46, wherein the disease condition is selected from the group consisting of cancer, hypertension, hypotension, ischemia, inflammation, arthritis, diabetic retinopathy, myocardial infarction, and cardiovascular disease.

48. The method according to claim 46, wherein the subject is human.

49. A method of treating a disease condition in a subject comprising:
providing an antibody or binding portion thereof according to claim 24;
contacting the antibody or binding portion thereof with a cell or tissue sample of a subject under conditions effective to bind the antibody or binding portion thereof to cells overexpressing IWU-1 from the cell or tissue sample; and
removing cells or tissues which bind to the antibody or binding portion thereof, thereby treating the disease condition.

50. The method according to claim 49, wherein the disease condition is selected from the group consisting of cancer, hypertension, hypotension, ischemia, inflammation, arthritis, diabetic retinopathy, myocardial infarction, and cardiovascular disease.

51. A method of treating a disease condition in a subject comprising:
providing a therapeutic amount of a pharmaceutical conjugate comprising:
an antibody or binding portion thereof according to claim 24 and a cytotoxic component; and
administering the conjugate to a subject under conditions effective to form an immune complex with any IWU-1 protein or polypeptide, thereby treating the disease condition.

52. The method according to claim 51, wherein the disease condition is selected from the group consisting of cancer, hypertension, hypotension, ischemia, inflammation, arthritis, diabetic retinopathy, myocardial infarction, and cardio-vascular disease.

53. The method according to claim 51, wherein the subject is human.

54. The method according to claim 51, wherein the administering is carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane.

55. A vaccine comprising:
a polypeptide or protein according to claim 21 or an antigenic fragment thereof.

56. A method of treating a disease condition in a subject comprising:
providing a composition comprising:
a polypeptide or protein according to claim 21, or antigenic fragment thereof; and
administering a therapeutically effective amount of the composition to a subject, thereby treating the disease condition.

57. The method according to claim 56, wherein the disease condition is selected from the group consisting of cancer, hypertension, hypotension, ischemia, inflammation, arthritis, diabetic retinopathy, myocardial infarction, and cardiovascular disease.

58. The method according to claim 56, wherein the subject is human.

59. A method of regulating IWU-1 expression in a subject comprising:
administering to the subject the antisense nucleic acid molecule according to claim 13.

60. A method of regulating IWU-1 expression in a subject comprising:
administering to the subject the nucleic acid molecule according to claim 1.

61. A method of regulating IWU-1 expression in a subject comprising:
administering to the subject a nucleic acid molecule according to claim 17.

62. A method of gene therapy for treating a disease condition comprising:
administering to a subject the nucleic acid molecule according to claim 1, thereby treating the disease condition of the subject.

63. The method according to claim 62, wherein said administering is carried out orally, parenterally, subcutaneously, intravenously, intramuscularly, intraperitoneally, by intravesical instillation, by intracavitary, intravesical instillation, intraocularly, intraarterially, intralesionally, or by application to mucous membrane.

64. The method according to claim 62, wherein the disease condition is selected from the group consisting of cancer, hypertension, hypotension, ischemia, inflammation, arthritis, diabetic retinopathy, myocardial infarction, and cardio-vascular disease.

65. The method according to claim 62, wherein the subject is a mammal.

66. The method according to claim 65, wherein the mammal is human.

67. A transgenic animal having an altered expression of IWU-1 protein or polypeptide, wherein the altered expression results from the introduction of a nucleic acid molecule according to claim 1 into the animal.

68. A transgenic animal according to claim 67, wherein the altered expression of IWU-1 protein or polypeptide is an up-regulation of the expression of IWU-1 protein or polypeptide.

69. A transgenic animal according to claim 67, wherein the altered expression of IWU-1 protein or polypeptide is a down-regulation of the expression of IWU-1 protein or polypeptide.

70. The transgenic animal according to claim 69, wherein the altered expression of IWU-1 results from the introduction of the antisense nucleic acid molecule according to claim 13 into the animal.

71. The transgenic animal according to claim 69, wherein the altered expression of IWU-1 protein or polypeptide results from RNA-interference.

72. A method of screening drugs that regulate IWU-1 activity comprising:
providing the IWU-1 protein or polypeptide according to claim 21;
providing a reagent upon which IWU-1 exerts activity;
providing a test compound;
combining the IWU-1 protein or polypeptide, the reagent, and the test compound in a mixture;
determining the activity of IWU-1 upon the reagent in the mixture; and
measuring any difference between the activity of IWU-1 upon the reagent with and without the test compound.

73. A method of screening drugs that regulate IWU-1 expression comprising:
transforming a host cell with a nucleic acid molecule according to claim 1 encoding an IWU-1 protein or polypeptide;
culturing the transformed cells;
adding a test compound to the culture containing the transformed cells; and
determining whether the test compound regulates expression of IWU-1 in the transformed cells.

74. The method according to claim 73, wherein the host cell is selected from the group consisting of a bacterial cell, a virus, a yeast cell, an insect cell, and a mammalian cell.

75. The method according to claim 73, wherein the host cell is a mammalian cell.

76. A method of screening drugs that regulate IWU-1 expression comprising:
isolating cells from a transgenic animal having an altered expression of IWU-1, wherein the altered expression results from the introduction of a nucleic acid molecule according to claim 1 into the animal;
adding a test compound to the isolated cells; and
determining whether the test compound regulates the expression of IWU-1 in the isolated cells.


SEQUENCE LISTING (16 sequences)

SEQ ID NO: 1 - DNA, 347 bp, Human
atgtgggagaggggtttcttccagatgcagactctcagtaatatcccttgtttctgcctccatggctcactcctgccctccactgatctggccactctctcagctcatcccactgatgacaccacgacgctctctgagagaccatccccaagcacagacgtccagacagacccccagaccctcaagccatctggttttcatgaggatgaccccttcttctatgatgaacacaccctccggaaacgggggctgttggtcgcagctgtgctgttcatcacaggcatcatcatcctcaccagtggcaagtgcaggcagctgtcccggttatgccggaatcattgcaggtg

SEQ ID NO: 2 - protein, 115 aa, Human [sequence omitted]

SEQ ID NO: 3 - protein, 36 aa, Human
DPFFYDEHTLRKRGLLVAAVLFITGIIILTSGKCRQ

SEQ ID NO: 4 - protein, 4 aa, Mouse
NYDT

SEQ ID NO: 5 - protein, 20 aa, Human
YYEVNGIFGLAVLLLLRRFC

SEQ ID NO: 6 - protein, 18 aa, Human
SYWSQVGICGCAMVMAKC

SEQ ID NO: 7 - protein, 18 aa, Human
TYQSQIGVIGILLVLRRC

SEQ ID NO: 8 - protein, 22 aa, Rat
SYWESQLGMIFGGLCAAMALKC

SEQ ID NO: 9 - protein, 47 aa, Human
MQTLSNIPCFCLHGSLLPSTDLATLSAHPTDDTTTLSERPSPSTDVQ

SEQ ID NO: 10 - protein, 46 aa, Human
LQLLKYIPPKAKSHSNLSTKMSTLSYRPSDNVSSSTKKPAPCFEVE

SEQ ID NO: 11 - protein, 46 aa, Human; identical to SEQ ID NO: 10

SEQ ID NO: 12 - DNA, 18 bp, Artificial Sequence
  Description of Artificial Sequence  Primer
aatctagagctccagcag

SEQ ID NO: 13 - DNA, 25 bp, Artificial Sequence
  Description of Artificial Sequence  Primer
tctcagtaatatcccttgtttctgc

SEQ ID NO: 14 - DNA, 23 bp, Artificial Sequence
  Description of Artificial Sequence  Primer
gttgtcagctcctgtttctgatg

SEQ ID NO: 15 - DNA, 647 bp, Human
tgcacaccagctgtgtagatgtgggagaggggtttcttccagatgcagactctcagtaatatcccttgtttctgcctccatggctcactcctgccctccactgatctggccactctctcagctcatcccactgatgacaccacgacgctctctgagagaccatccccaagcacagacgtccagacagacccccagaccctcaagccatctggttttcatgaggatgaccccttcttctatgatgaacacaccctccggaaacgggggctgttggtcgcagctgtgctgttcatcacaggcatcatcatcctcaccagtggcaagtgcaggcagctgtcccggttatgccggaatcattgcaggtgagtccatcagaaacaggagctgacaacccgctgggcacccgaagaccaagccccctgccagctcaccgtgcccagcctcctgcatcccctcgaagagcctggccagagagggaagacacagatgatgaagctggagccagggctgccggtccgagtctcctacctcccccaaccctgcccgcccctgaaggctacctggcgccttgggggctgtccctcaagttatctcctctgttaagacaaaaagtaaagcactgtggtctttgcaaaaaaaaaaaaaaa

SEQ ID NO: 16 - protein, 14 aa, Artificial Sequence
  UNSURE (2): Xaa at position 2 can be any amino acid
DXFXYDXXSLRXXG